(19) United States
(12) Patent Application Publication
(10) Pub. No.: US 2022/0105318 A1
(43) Pub. Date: Apr. 7, 2022

(54) MICROFABRICATED CORE WIRE FOR AN INTRAVASCULAR DEVICE

(71) Applicant: SCIENTIA VASCULAR, LLC, West Valley City, UT (US)

(72) Inventors: Clark C. Davis, Holladay, UT (US); John A. Lippert, Park City, UT (US); William Rigby Eccles, Centerville, UT (US)

(21) Appl. No.: 17/493,281

(22) Filed: Oct. 4, 2021

Related U.S. Application Data
(60) Provisional application No. 63/087,411, filed on Oct. 5, 2020.

(51) Int. Cl. A61M 25/09; A61M 25/00

(52) U.S. Cl. CPC A61M 25/09; A61M 2025/09075; A61M 2025/0042; A61M 25/0021

(57) ABSTRACT

This disclosure describes microfabricated core wires configured for use in an intravascular device such as a guidewire or microcatheter. A microfabricated core wire includes a plurality of disks spaced apart from one another along a length of the distal section and a plurality of longitudinally extending ribbons interposed between the disks, each ribbon extending between and connecting a pair of adjacent disks. Various arrangements of disks and ribbons may be provided to control stiffness across a length of the core wire, particularly at or near the distal tip of the core wire.

### CROSS-REFERENCE TO RELATED APPLICATIONS

This application claims priority to and the benefit of U.S. Provisional Patent Application Ser. No. 63/087,411, filed Oct. 5, 2020, and titled “Microfabricated Core Wire for an Intravascular Device.” The entirety of the foregoing application is incorporated herein by this reference.

### BACKGROUND

Guidewires are often used to lead or guide catheters or other interventional devices to a targeted anatomical location within a patient's body. Typically, guidewires are passed into and through a patient's vasculature in order to reach the target location, which is often at the patient's heart or brain, for example. Radiographic imaging may be utilized to assist in navigating the guidewire to the targeted location. In many instances, the guidewire is placed within the body and is then used to guide one or more catheters or other interventional devices to the targeted anatomical location for the delivery of drugs, stents, embolic coils, or other substances or devices for treating the patient.

In many applications, the guidewire must be routed through the tortuous bends and curves of a vasculature passageway to arrive at the targeted anatomy. For example, directing the guidewire to portions of the neurovasculature requires passage through the internal carotid artery and other tortuous paths. Accordingly, such guidewires require sufficient flexibility, particularly at distal portions, to navigate effectively. However, other design aspects must also be considered. For example, the guidewire must also be able to provide sufficient torquability (i.e., the ability to transmit torque applied at the proximal end all the way to the distal end), pushability (i.e., the ability to transmit axial push to the distal end rather than bending and binding intermediate portions), and structural integrity for performing intended medical functions.

Some guidewires are constructed with a core wire (often simply referred to as the core) and an outer tube that surrounds a distal portion of the core. The outer member may also include machined slots or fenestrations to increase its bending flexibility. The intent behind such designs is to reduce the diameter of the core in the distal sections of the guidewire in order to increase the flexibility of the core, while utilizing the larger outer diameter of the outer member for torque transmission.

While such guidewires have been successful, several limitations remain. For example, the core wires of such devices contribute to the axial stiffness of the device, which is beneficial for enabling good “pushability” and linear control. However, if the distal end of the guidewire comes into contact against tissue and the core wire has excessive columnar stiffness, tissue could be injured.

The use of a core wire and a surrounding tube also creates an annular space between the outer surface of the core and the inner surface of the tube. When the guidewire navigates a bend, the core may move out of alignment with the center line of the tube. Such off-centering can disrupt the smooth distal transmission of rotational movement, and can cause a buildup and sudden release of forces resulting in unwanted “snap” and/or “whip” movements. This disruption to the tactile feel and control of the guidewire can make it more difficult for the operator to rotationally position the guidewire as intended, raising the risk of interventional procedure delays, suboptimal outcomes, inability to access the target location, or even tissue injury.

Accordingly, there is an ongoing need for improved guidewires and other intravascular devices. In particular, there is an ongoing need for intravascular devices having core wires optimized to enhanced benefits relative to devices with conventional core wires.

### SUMMARY

The present disclosure describes microfabricated core wires configured for use in an intravascular device such as a guidewire or microcatheter. A microfabricated core wire includes a plurality of disks spaced apart from one another along a length of the distal section and a plurality of longitudinally extending ribbons interposed between the disks, each ribbon extending between and connecting a pair of adjacent disks. Various arrangements of disks and ribbons may be provided to control stiffness across a length of the core wire, particularly at or near a distal section of the core wire.

In one embodiment, the disks and ribbons are arranged to form a preferred bending plane in the distal section of the core wire, or to form multiple preferred bending planes in the distal section of the core wire. For example, the disks and ribbons are arranged so as to enable the distal section to form a compound curve.

In one embodiment, a distal section of the core wire includes a first portion and a second portion that is distal of the first portion, wherein the first portion is configured to provide a first preferred bending plane, and the second portion is configured to provide a second preferred bending plane different from the first preferred bending plane. In some embodiments, the first and second preferred bending planes are substantially orthogonal to one another

In one embodiment, a core wire may include an arrangement of disks and ribbons where the ribbons have any combination of: a rotational offset; a height placement offset; variable ribbon height; variable ribbon width; multiple sections associated with different preferred bending planes; and intentional shaping ribbons

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1 through 2B illustrate an example of a guidewire device having a conventional core wire;

FIG. 3 illustrates a core wire having disks and ribbons arranged to form a preferred bending plane;

FIG. 4A illustrates a core wire having disks and ribbons arranged with a 90 degree rotational offset to form multiple preferred bending planes;

FIG. 4B illustrates a core wire having disks and ribbons arranged with a rotational offset that provides a helical pattern along the length of the core wire;

FIG. 5 illustrates a core wire having disks and ribbons arranged with ribbons of differential height;

FIG. 6 illustrates a core wire having disks and ribbons arranged with ribbons of differential width;

FIG. 7 illustrates a core wire having disks and ribbons arranged that are not aligned along the longitudinal axis of the core wire but are instead aligned along one side of the core wire;

FIG. 8 illustrates a core wire having disks and ribbons arranged such that the ribbons have differential height placement and form a “slant” profile;

FIG. 9 illustrates a core wire having disks and ribbons arranged such that the ribbons have differential height placement and form a “serpentine” profile;

FIG. 10 illustrates a core wire having disks and ribbons arranged in multiple regions with multiple corresponding preferred bending planes; and

FIG. 11 illustrates a core wire having disks and ribbons arranged with a shaping ribbon that has greater predisposition to bending relative to the ribbons proximal and distal of the shaping ribbon.

### DETAILED DESCRIPTION

### Introduction

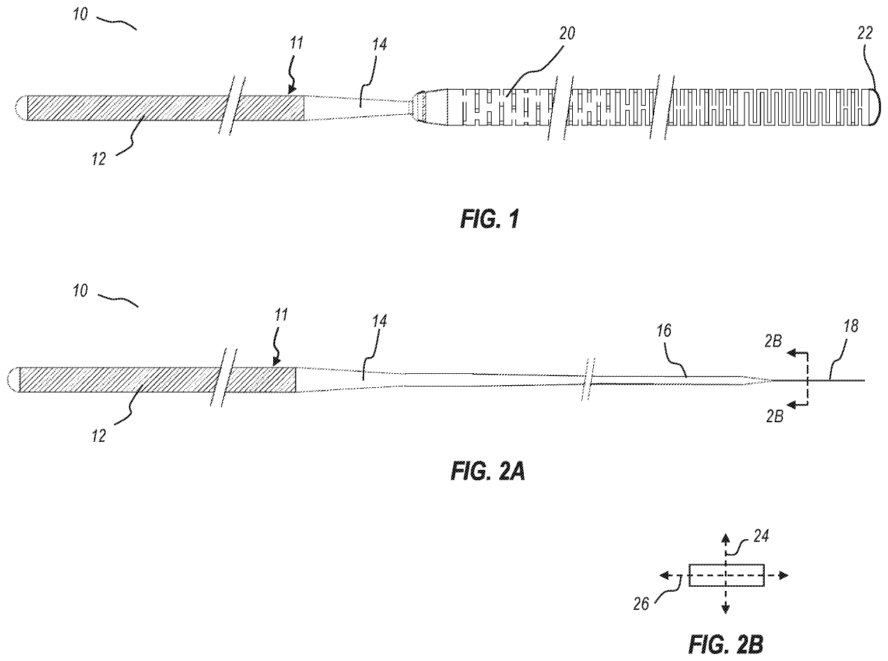

FIGS. 1 through 2B illustrate an example guidewire device 10 that includes a conventional core wire 11. In FIG. 2A, the tube 20 has been removed to more clearly show the distal portions of the core wire 11. The conventional core wire 11 has a proximal section 12, one or more transition sections 14 where the diameter narrows as compared to the proximal section 12, and a first distal section 16 disposed within the tube 20. The core wire 11 may be made from any suitable material such as stainless steel or other metals or alloys having similar material properties.

The tube 20 may be microfabricated to include various cutting patterns to control flexibility of the tube 20. The tube 20 extends to an atraumatic distal tip 22, which often comprises a polymer material. The tube itself may be made from any suitable material. A highly flexible or superelastic material, such as nitinol or other materials with similar properties, are suitable.

The overall length of the guidewire device 10 may vary according to application needs, but typically ranges from about 200 cm to about 300 cm. The length of the microfabricated tube 20 may also vary according to particular application needs, but will typically range from about 25 cm to about 45 cm.

Often, a second distal section 18 of the core wire 11 is flattened and has a rectangular cross-sectional shape, as shown in the cross-sectional view of FIG. 2B. A flattened distal tip provides good flexibility along a plane 24 orthogonal to the long dimension of the rectangular cross-section, but remains relatively stiff in bending along a plane 26 parallel to the long dimension of the rectangular cross-section.

The core wire 11 provides beneficial internal structure and axial stiffness to distal sections of the guidewire device 10. However, too much stiffness in the core wire 11 may be detrimental. For example, if the core wire 11 has too much column stiffness, particularly at distal sections of the core wire 11, then bumping the distal end of the guidewire against tissue could cause injury to the tissue. This could happen during navigation of the guidewire through the vasculature, for example. Put another way, if the column stiffness of the core wire 11 is too high, the distal tip of the device will impinge and/or puncture contacted tissue before the core wire laterally deflects under the applied load.

Guidewire devices such as guidewire device 10 also typically have an annular space between the outer surface of the core wire 11 and the inner surface of the tube 20. Because of the annular space, when the guidewire device navigates a bend, the core wire 11 may move out of alignment with the center line of the tube 20. Such off-centering can disrupt the smooth distal transmission of rotational movement, and can cause a buildup and sudden release of forces resulting in undesirable “snap” and/or “whip” movements.

For a given diameter of tube 20, simply increasing the diameter or cross-sectional size of the core wire 11 to better fill the annular space is not a practical option because doing so would make the distal sections of the core wire 11 much too stiff. In other words, though making the core wire 11 larger in distal sections of the guidewire may provide better centering of the core wire 11 within the tube 20, it likely also results in excessive bending stiffness, column stiffness, or both.

As one example, a guidewire core 11 may be ground down to about 0.002 inches in diameter near the distal end. Flattening the distal end 18 of the core provides a width of about 0.003 inches in the longest dimension. The inner diameter of the tube 20 may be about 0.008 inches. This means that the ratio of the outer diameter or size (i.e., size in longest cross-sectional dimension) of the distal section of the core wire to the inner diameter of the tube is about 0.25 or 0.375. For larger guidewire sizes, the core wire typically has similar distal dimensions because of the practical limits on diameter/size required to avoid excessive stiffness. Thus, for larger guidewire sizes with larger tube diameters (e.g., 0.018 inches, 0.024 inches), the ratios of core diameter/size to tube inner diameter are even lower.

A lower ratio means there is more annular space to be filled and thus more potential for the core wire to become axially misaligned with the tube at bends. As mentioned above, increasing the diameter/size of the core risks overly increasing the stiffness (bending and/or column) of the core. However, the microfabricated core wires disclosed herein can be provided with a relatively larger diameter/size. The microfabricated structure of the core provides additional bending flexibility and/or reduced column stiffness that allows the use of larger diameters/sizes without the usual associated problems of excessive stiffness.

For example, an intravascular device (e.g., guidewire) utilizing a microfabricated core wire as disclosed herein may include a tube such as shown in FIG. 1, and the core wire and tube may be sized such that the ratio of the outer diameter/size of the distal section of the core wire to the inner diameter of the tube is greater than about 0.375, preferably about 0.5 or greater, more preferably about 0.625 or greater.

Thus, for an intravascular device having a tube with an inner diameter of about 0.008 inches, the core wire may have a diameter/size greater than 0.002 inches or greater than about 0.003 inches, such as about 0.0035 inches to about 0.008 inches, or about 0.004 inches to about 0.006 inches, or within a range defined by endpoints selected from any two of the foregoing values. Where the inner diameter of the tube is a different size, these values may be adjusted proportionally.

For purposes of determining the above ratios, the diameter/size of the core wire should include at least the distal most 1 to 3 cm of the core wire. Providing relatively larger sizes at the very distal regions of the core while maintaining acceptable flexibility and performance characteristics at these distal regions represents an advancement over conventional intravascular devices. That is, a standard guidewire core may have a larger diameter/size at more proximal regions of the device, but not at the more distal regions of the device.

### Microfabricated Core Wires

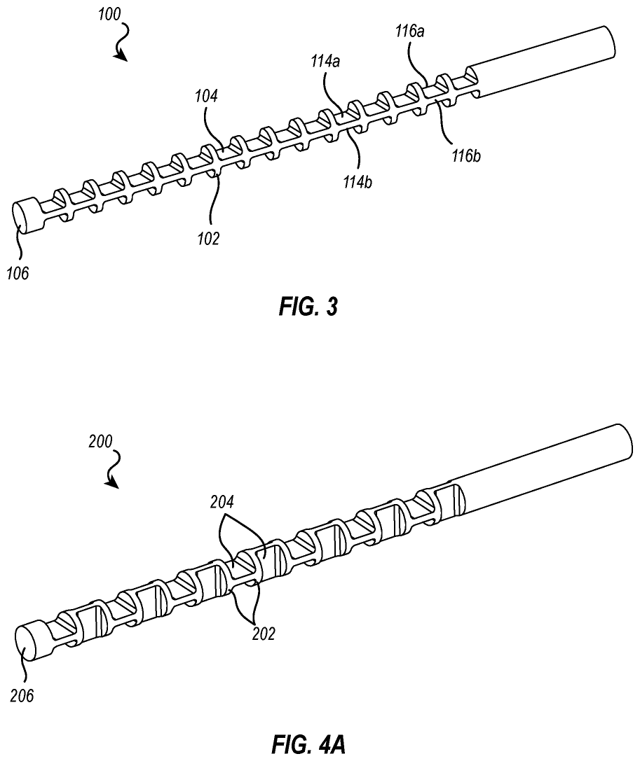

FIG. 3 illustrates an exemplary core wire 100 extending between a proximal end (not shown) and a distal end 106. A distal section of the core wire 100 is microfabricated to form a plurality of disks 102 spaced apart from one another along a length of the distal section with a plurality of longitudinally extending ribbons 104 interposed between the disks 102. Each ribbon 104 extends between and connects a pair of adjacent disks.

The disks 102 and ribbons 104 may be formed using any suitable microfabrication method, including cutting or other micro-machining techniques and/or laser ablation/cutting techniques (e.g., using a femtosecond laser). Machining or ablating away some of the stock material of the core wire forms the ribbons 104 while the disks 102 are defined between each “cut” of the stock material. The arrangement of disks 102 and ribbons 104 can be beneficially tailored to provide desired flexibility characteristics of the core wire 100. As compared to a similarly sized core wire that has not been microfabricated, the microfabricated core wire 100 can be made to be significantly more flexible.

The length of the microfabricated distal section of the core wire 100 may vary according to particular application needs. In some embodiments, it may have a length of at least about 0.5 cm up to about 1 cm, or up to about 3 cm, or up to about 5 cm, or up to about 7.5 cm, or up to about 10 cm, or up to about 15 cm, or up to about 20 cm, or up to about 25 cm, or up to about 30 cm, or up to about 35 cm.

The disks 102 and ribbons 104 may be configured with various different sizes and shapes to provide different characteristics to the core wire 100. For example, spreading out the “cuts” (used herein to include cuts, ablations, or other subtractive machining processes) of the core wire 100 so that there is greater space between each ribbon 104 will make the resulting disks 102 longer. The length of a disk is the distance from one side of a disk to the other in a longitudinal direction. Longer disks 102 and greater spacing between ribbons 104 results in greater stiffness than shorter disks 102 with less spacing between ribbons 104, all else being equal. Each disk also has a disk width, defined as the diameter of the planer face of the disk. This disk width may be equal to the width of the core wire 100. The disk width may also vary according to the needs of the application and different disk widths may be used for different sections of the core wire (e.g., the distal section may have a narrower disk width than more proximal sections).

The length of ribbons 104 (distance from one disk to the next in the longitudinal direction) may also be adjusted. Longer ribbons 104 provide more flexibility than shorter ribbons 104, all else being equal. Each ribbon 104 also has a width defined as the distance between lateral face 116a to lateral face 116b. Narrower ribbons provide more flexibility than wider ribbons, all else being equal. Each ribbon 104 also has a height defined as the distance between planar face 114a to planar face 114b. The height of the ribbons can be controlled by adjusting the depth of the cut made into the material and thus the amount of material removed to form the ribbon. Ribbons with shorter height provide more flexibility than ribbons with greater height, all else being equal.

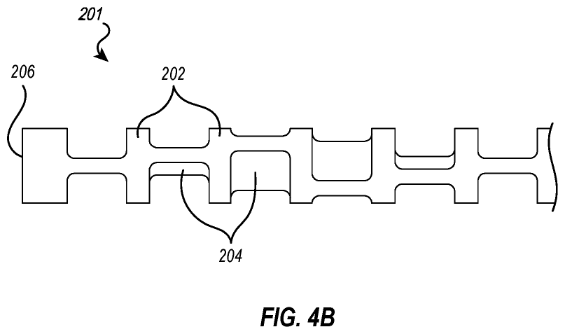

In some embodiments, the disks 102 and ribbons 104 are arranged to form a preferred bending plane in the distal section of the core wire 100. As shown, because multiple ribbons 104 are aligned within the same general plane, the core wire 100 will have a preferred bending plane orthogonal to the width of the ribbons 104 while resisting bending in the plane parallel to the width of the ribbons 104. Other embodiments may be configured to provide additional bending planes or to avoid the formation of any bending planes (see, e.g., FIG. 4B). Core wires may therefore be configured according to particular application needs and preferences.

FIG. 4A illustrates a core wire 200 extending from a proximal end to a distal end 206 and having disks 202 and ribbons 204 arranged to form two preferred bending planes. This is accomplished by rotationally offsetting some of the ribbons 204 relative to other ribbons 204. In this example, every successive ribbon 204 is rotationally offset by 90 degrees from the preceding ribbon. This provides the core wire 200 with two preferred bending planes that are substantially orthogonal to each other.

Other embodiments may utilize different rotational offsets. For example, the rotational offset may be more or less than 90 degrees so that when applied to multiple ribbons along a length of the core wire 200 the ribbons form a “helical” pattern that rotates about the circumference of the core wire as it moves along its length. A helical pattern minimizes preferred bending axes in the core wire 200. FIG. 4B illustrates one non-limiting example of a core wire 201 configured with a rotational offset of ribbons that provides a helical pattern.

Other beneficial “distributed” arrangements may alternatively be utilized to avoid preferred bending axes. These are described in more detail in International Patent Publication No. WO 2018/218216 A1, titled “Micro-Fabricated Medical Device Having a Non-Helical Cut Arrangement,” and which is incorporated herein by this reference in its entirety. WO 2018/218216 A1 describes various cut patterns mostly in the context of tubular members, and may be applied to any of the tubes used with the presently disclosed core wires. It is contemplated by the present disclosure that the same cut pattern principles described in WO 2018/218216 A1 may be applied to the core wires described herein.

The rotational offset is shown here as being applied to each successive ribbon 204. In other embodiments, a rotational offset may be applied less frequently, such as after a set of two or more ribbons (referred to herein as a “ribbon set”), or according to some other pattern. The rotational offset may be a constant value. Alternatively, a varying rotational offset (such as one that gets progressively greater or smaller toward the distal end 206) may be applied.

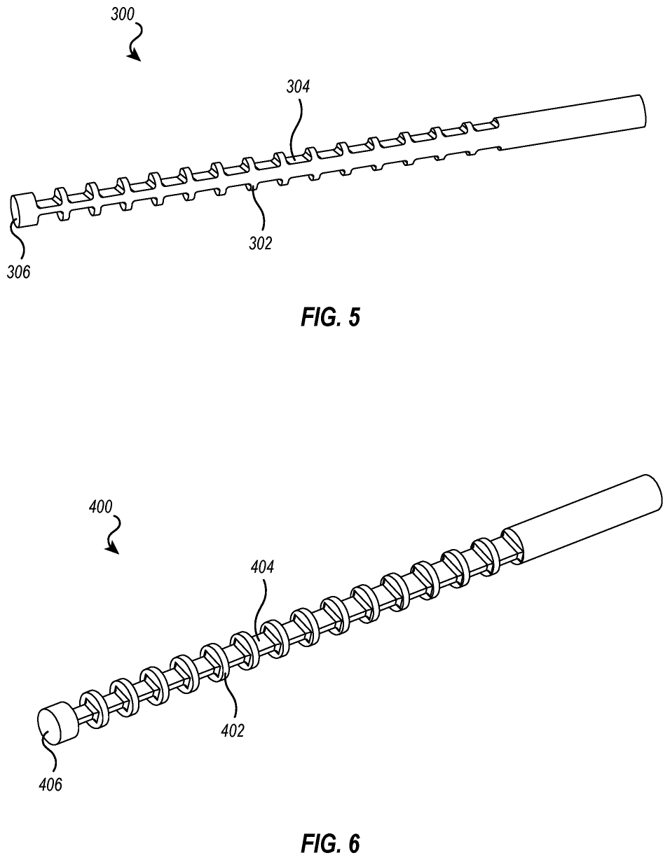

FIG. 5 illustrates an embodiment of a core wire 300 having disks 302 and ribbons 304 where the ribbons 304 do not all have the same height. As shown, proximal ribbons 304 have greater relative height and the ribbon height progressively shortens toward the distal end 306 of the core wire 300. This arrangement can make the flexibility of the core wire 300 progressively increase toward the distal end 306. Alternatively, the core wire 300 may have ribbons 304 that are not all the same height, but the change in height is not necessarily progressive.

FIG. 6 illustrates an embodiment of a core wire 400 with disks 402 and ribbons 404 where the ribbons 404 do not all have the same width. As shown, proximal ribbons 404 have greater relative width and the ribbon width progressively narrows toward the distal end 406 of the core 400. This arrangement can make the flexibility of the core wire 400 progressively increase toward the distal end 406. Alternatively, the core wire 300 may have ribbons 304 that are not all the same width, but the change in width is not necessarily progressive.

Adjusting the widths of the ribbons 404 may be accomplished by forming cuts on lateral surfaces of the ribbons 404 in addition to the cuts that form the planar surfaces. In this way, the lateral surfaces of the ribbons 404 do not necessarily align with the circumference of the adjacent disks 402. The extra space resulting from these lateral cuts can also beneficially allow room for placement of a marker band on or around the ribbon 404. The marker band may be made of a radiopaque material (e.g., platinum or other material more radiopaque than stainless steel).

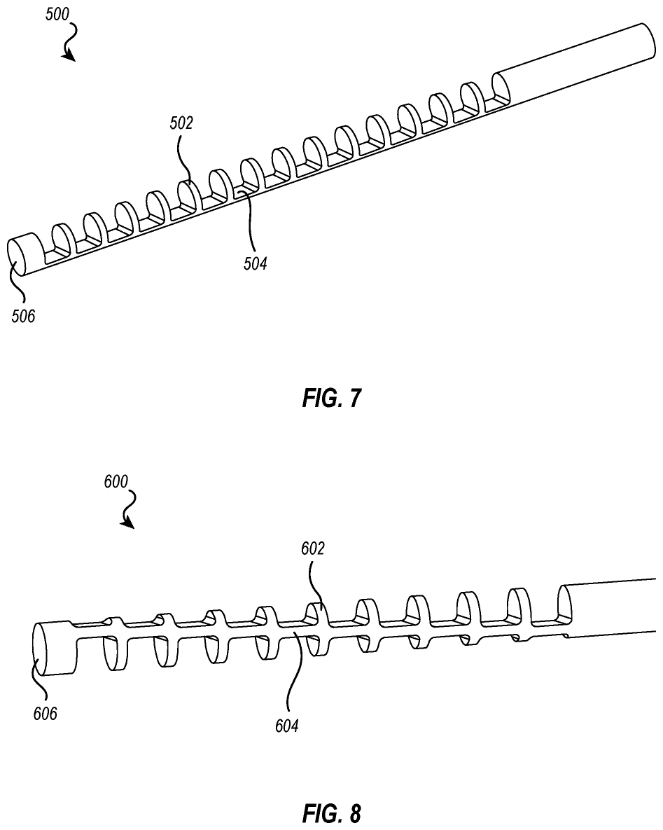

FIG. 7 illustrates an embodiment of a core wire 500 extending from a proximal end to distal end 506 and having an arrangement of disks 502 and ribbons 504. In this embodiment, the ribbons 504 are not centered with the longitudinal axis of the core wire 500. In this particular embodiment, each ribbon 504 is aligned on the same side of the core wire 500. In other words, each ribbon 504 is aligned along a line that is parallel to the longitudinal axis of the core wire 500 but is closer to an outer circumference of the core wire 500 than to the longitudinal axis, which runs through the center of the core wire 500.

FIG. 8 illustrates an embodiment of a core wire 600 extending from a proximal end to distal end 606 and having an arrangement of disks 602 and ribbons 604. As with core wire 500, not all of the ribbons 604 of core wire 600 are centered with the longitudinal axis of the core wire 600. However, unlike core wire 500, the ribbons 604 of core wire 600 have varying height placements rather than being aligned along a single line at a single height. In other words, the height position of each ribbon 604 between its corresponding pair of adjacent disks 602 may vary.

In the illustrated embodiment, a height offset is applied to each successive ribbon 604 along a length of the core wire 600, resulting in a “slanted” arrangement of ribbons 604. The height offset may be adjusted to make the slant(s) of the core wire 600 shallower or steeper. The height offset may be applied between each successive ribbon 604, as shown, or may be applied between ribbon sets. The height offset may be a constant value. Alternatively, a varying height offset (such as one that gets progressively greater or smaller toward the distal end 606) may be applied. The result of the successive height offset is a slanted profile, as illustrated in FIG. 8.

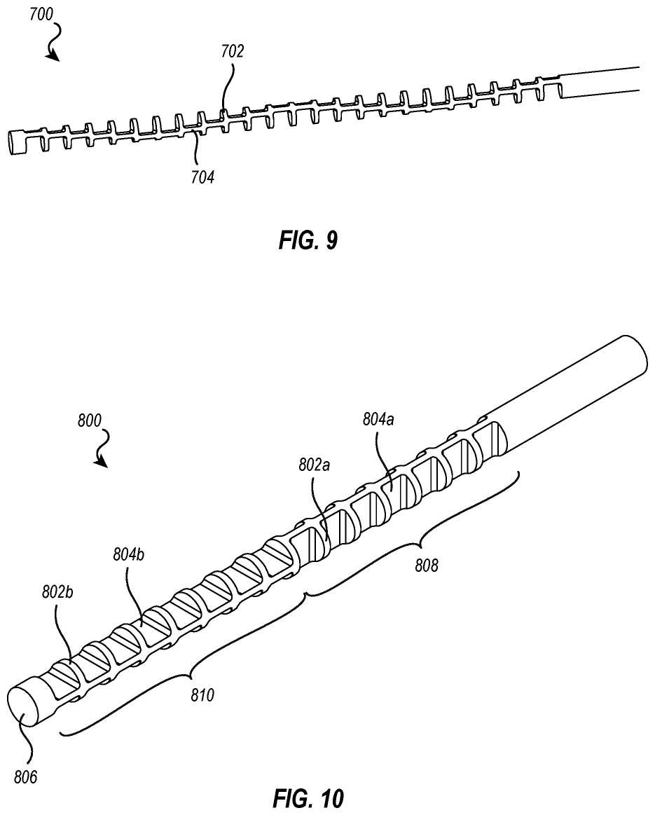

FIG. 9 illustrates an embodiment of a core wire 700 that is similar to core wire 600. Here, core wire 700 includes disks 702 and ribbons 704 with the ribbons 704 are positioned at varying heights according to a height offset between ribbons 704. Rather than a single slant pattern, the height offset reverses direction one or more times to form a “serpentine” arrangement of ribbons 704. The result of the successive height offset where the direction of the offset reverses one or more times, is a serpentine profile, as illustrated in FIG. 9.

Generally, embodiments such as those in FIGS. 7 through 9 may be described as having one or more ribbons positioned such that a longitudinally extending centerline of the ribbon is out of alignment with a longitudinally extending centerline of the core wire (i.e., the longitudinal axis of the core wire). Such embodiments provide asymmetric/eccentric structures with reduced column stiffness. Such embodiments tend to deflect under comparatively lighter axial loads as a result of the intentional misalignment between the ribbons and the longitudinal axis of the core wire. The reduced column stiffness of these embodiments is beneficial in certain applications, particularly where there is risk of the distal tip running into vascular tissue or other anatomy during a procedure.

FIG. 10 illustrates an embodiment of a core wire 800 extending from a proximal end to a distal end 806 and having multiple sections each with different arrangements of disks 802 and ribbons 804. In the illustrated embodiment, a first (proximal) region 808 includes disks 802a and ribbons 804a arranged to provide a first preferred bending plane, and a second (distal) region 810 includes disks 802b and ribbons 804b arranged to provide a second, different bending plane. The first and second bending planes may be substantially orthogonal to each other, as in the illustrated embodiment, or may be at some other transverse angle.

Because the separate bending planes are associated with discrete, separate regions of the core wire 800, the separate bending planes allow the core wire 800 to be bent and/or shaped into a compound curve. This can beneficially enable the surgeon to put the distal tip of an intravascular device into multiple orientations by utilizing one or more of the bending planes in addition to selectively rotating the intravascular device.

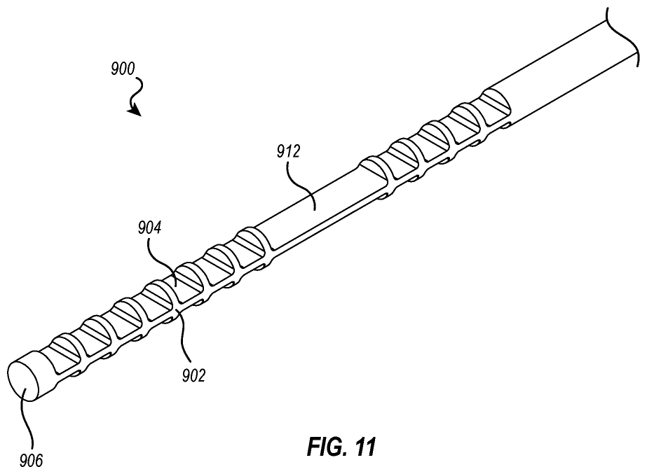

FIG. 11 illustrates an embodiment of a core wire 900 extending from a proximal end to a distal end 906 and having an arrangement of disks 902 and ribbons 904. The core wire 900 also includes a shaping ribbon 912 configured to be an intentional “weak spot” where the user can easily form a bend, such as a relatively sharp bend to make a “hockey stick” shape at the distal tip. The shaping ribbon 912 is preferably configured to have greater susceptibility to bending than more proximal ribbons and more distal ribbons so as to form the intended bend point. The greater susceptibility to bending may be provided by making the shaping ribbon 912 with one or more of greater length, smaller width, or smaller height than the more proximal ribbons and more distal ribbons.

The exemplary microfabricated core wires shown in FIGS. 3 through 11 illustrate various different possible arrangements of disks and ribbons. However, embodiments need not be limited to the particular arrangements shown. Features from one or more embodiments may be combined with different features from one or more different embodiments. For example, a core wire may include ribbons with any combination of: a rotational offset; a height placement offset; variable ribbon height; variable ribbon width; multiple sections associated with different preferred bending planes; and intentional shaping ribbons.

Any of the microfabricated core wires described herein may be utilized in an intravascular device such as a guidewire. For example, any of the microfabricated core wires described herein may replace the core wire 11 of FIG. 1 and be combined with the tube 20 to form an improved guidewire device. The tube may be coupled to the guidewire on distal end of the guidewire, or a proximal end of a guidewire, or both, for example. In some embodiments, where the tube is also microfabricated and has one or more preferred bending planes, the core wire is configured and positioned so that one or more preferred bending planes of the core wire align with the one or more preferred bending planes of the tube. For example, a tube may have a plurality of circumferentially extending rings and axially extending beams arranged to provide one or more preferred bending planes to the tube. The core wire will be disposed within and aligned with the tube to align the one or more preferred bending planes of the tube and with the preferred bending planes of the core. In this manner, the preferred bending planes of the tube and the core wire work in concert with each other.

In some embodiments, the difference between the outer diameter of the core wire and the inner diameter of the tube will be optimized to improve the annular space between the core wire and the tube. In some embodiments, the ratio of the outer diameter of the core wire to the inner diameter of the tube is greater than about 0.375, preferably about 0.5 or greater, more preferably about 0.625 or greater.

Although the microfabricated core wires disclosed herein can be made larger in diameter/size at distal sections than conventional core wires, and thereby better fill the annular space between the core wire and the tube, there may still be some amount of annular space remaining. Various additional centering mechanisms, including one or more coils, polymer fillers, and/or tubes, may be provided to help further fill the annular space and further enhance centering. Such centering mechanisms are described in greater detail in U.S. patent application Ser. No. 16/742,211, titled “Guidewire with Core Centering Mechanisms,” and which is incorporated herein by this reference in its entirety.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A core wire configured for use in an intravascular device, the core wire extending between a proximal end and a distal end, at least a distal section of an elongated member being microfabricated so as to include:
a plurality of disks spaced apart from one another along a length of the distal section; and
a plurality of longitudinally extending ribbons interposed between the disks, each ribbon extending between and connecting a pair of adjacent disks.

2. The core wire of claim 1, wherein the microfabricated distal section of the core wire has a length of at least about 0.5 cm up to about 35 cm.

3. The core wire of claim 1, wherein the distal section of the core wire has an outer diameter of about 0.003 inches to about 0.010 inches.

4. The core wire of claim 1, wherein the disks and ribbons are arranged to form one or more preferred bending planes in the distal section of the core wire.

5. The core wire of claim 4, wherein the distal section of the core wire includes a first portion and a second portion that is distal of the first portion, wherein the first portion is configured to provide a first preferred bending plane, and the second portion is configured to provide a second preferred bending plane different from the first preferred bending plane.

6. The core wire of claim 5, wherein the first and second preferred bending planes are substantially orthogonal to one another.

7. The core wire of claim 1, wherein at least one ribbon is disposed such that it has a rotational offset relative to another ribbon.

8. The core wire of claim 7, wherein the rotational offset is applied to successive ribbons or ribbon sets along a length of the distal section of the core wire so that each successive ribbon or ribbon set has a different rotational placement than its preceding ribbon or ribbon set.

9. The core wire of claim 1, wherein at least one ribbon has a height that is different from at least one other ribbon.

10. The core wire of claim 9, wherein ribbon height progressively decreases toward the distal end of the core wire.

11. The core wire of claim 1, wherein at least one ribbon is laterally cut on one or both lateral sides so as to have a ribbon width that is less than a diameter of one or both adjacent disks.

12. The core wire of claim 11, wherein the ribbon width progressively narrows toward the distal end of the core wire.

13. The core wire of claim 1, wherein at least one ribbon has a height placement between its adjacent disks such that a longitudinally extending centerline of the ribbon is out of alignment with a longitudinally extending centerline of the core wire.

14. The core wire of claim 13, wherein a height offset is applied to successive ribbons or ribbon sets along a length of the distal section of the core wire so that each successive ribbon or ribbon set has a different height placement than its preceding ribbon or ribbon set.

15. The core wire of claim 14, wherein the ribbons are arranged to form a slanted profile and/or a serpentine profile along a portion of the microfabricated distal section of the core wire.

16. The core wire of claim 1, wherein at least one ribbon has greater susceptibility to bending than more proximal ribbons and more distal ribbons so as to form an intended bend point at the at least one ribbon.

17. An intravascular device, comprising:
a tube; and
a core wire extending between a proximal end and a distal end, the core wire including
a plurality of disks spaced apart from one another along a length of a distal section, and
a plurality of longitudinally extending ribbons interposed between the disks, each ribbon extending between and connecting a pair of adjacent disks.

18. The intravascular device of claim 17, wherein the tube has an inner diameter, and wherein a ratio of an outer diameter or size of the distal section of the core wire to the inner diameter of the tube is greater than about 0.375.

19. The intravascular device of claim 17, wherein the tube is microfabricated and has a plurality of circumferentially extending rings and axially extending beams arranged to provide one or more preferred bending planes to the tube, and wherein the core wire is positioned so that one or more preferred bending planes of the tube are aligned with one or more substantially similar bending planes of the core wire.

20. A method of microfabricating a stock wire which is configured for use in an intravascular device, including:
removing material of the stock wire to form a plurality of disks spaced apart from one another along a length of a distal section; and
forming a plurality of longitudinally extending ribbons interposed between the disks, each ribbon extending between and connecting a pair of adjacent disks and configured to provide flexibility and one or more preferred bending planes.

21. A guidewire, comprising:
a microfabricated tube comprising a plurality of circumferentially extending rings and a plurality of axially extending beams; and
a core wire extending between a proximal end and a distal end, the core wire comprising
a plurality of disks spaced apart from one another along a length of a distal section, and
a plurality of longitudinally extending ribbons interposed between the disks, each ribbon extending between and connecting a pair of adjacent disks.
